(19) United States
(12) Patent Application Publication
(10) Pub. No.: US 2002/0183532 A1
(43) Pub. Date: Dec. 5, 2002

(54) Bioavailable prodrugs of androgenic steroids and related method

(76) Inventor: William J. Roberts, Gainesville, FL (US)

Correspondence Address: Sullivan Law Group, Suite 120, 5060 North 40th Street, Phoenix, AZ, 85018-2140, US

(21) Appl. No.: 09/872,705

(22) Filed: Jun. 1, 2001

(51) Int. Cl. C07J 3/00; A61K 31/56; C07J 1/00

(52) U.S. Cl. 552/611; 552/638; 514/178

(57) ABSTRACT

A compound for supplementing the concentration of a parent androgen in a subject in vivo, wherein the parent androgen has a skeletal structure including a 17 position and the parent androgen further has a 17β-hydroxy group comprising a 17β-hydroxy hydrogen appended to the 17 position. The compound comprises a substrate having the skeletal structure of the parent androgen including a 17 position corresponding to the 17 position of the parent androgen, and a promoiety comprising an alkoxymethyl ether appended to the 17 position of the substrate as a substitute for the 17β-hydroxy hydrogen. Related compounds and methods also are disclosed.

### BACKGROUND OF THE INVENTION

1. Field of the Invention

The present invention pertains to the field of biochemistry and more specifically to the field of prodrugs or prohormones of androgenic steroids and related methods.

2. Description of the Related Art

The importance of having sufficient concentrations of androgenic steroids in the body is well known. The advantages of such steroids can include such things as increased physical performance, improved body composition or increased density, and increased sexual drive or performance, depending upon the specific steroid, organism, etc. It is equally well known that under many common circumstances, deficiencies of these androgenic steroids arise. In humans, for example, as one ages, the normal concentrations of these steroids tend to decrease. Men over the age of about thirty-five typically suffer a reduction in the blood serum concentration of free testosterone. These changes typically result in a reduced general athletic performance and longer time requirements for restoration after extensive exercise, as well as reduced physical and psychological resistance to stress.

A known approach to addressing such steroid concentration deficiencies is to introduce the deficient steroid into the body. In U.S. Pat. No. 5,578,588, for example, methods are disclosed for delivering testosterone supplements including peroral administration or intramuscular injection of testosterone.

This approach, however, suffers from a number of disadvantages. Introduction of such steroid supplements has been associated with undesirable effects such as poor control over blood concentrations and loss of the steroid due to a “first pass effect,” wherein the steroid is metabolized by the liver prior to reaching general circulation. These losses dramatically reduce the available steroid and, consequently, much higher doses of the steroid supplement generally must be administered to achieve the desired effects. The higher doses sometimes result in an undesirable and unpredictable rise in overall steroid concentration, which, for example, in the case of testosterone, can result in physiological and psychological problems.

Another approach to addressing steroid concentration deficiencies is to introduce a prodrug of the steroid into the body. An example of a prodrug is a prohormone. A prohormone is a compound that itself has no anabolic activity but, when administered in the body, is metabolized or converted into a natural or desired hormone. Such prohormones become substrates for in vivo bioconversion into the parent compounds, i.e., the corresponding natural or desired hormones. U.S. Pat. No. 5,053,403, for example, discloses that specific prohormones including androstenedione, progesterone, and 17α-β derivatives or analogues can be administered to humans for the purpose of increasing blood concentration of testosterone, with fewer undesirable effects. Long term use of these prohormones, however, is also associated with side effects, such as gynecomastia. A pernasal dose of 3.5 mg to 15 mg of these prohormones is reported to increase the blood concentration of testosterone by 34% to 97%. Similarly, U.S. Pat. No. 5,880,117 discloses the use of 4-androstenediol as a peroral testosterone supplement. Androstenedione is a direct precursor of testosterone and estrogen in target tissues having appropriate receptors and enzymes. According to U.S. Pat. No. 6,117,429, androstenediols are precursors for testosterone after oral administration on adults. 19-norandrostenedione is a precursor for 19-nortestosterone, which has a similar anabolic activity in comparison to testosterone.

The general approach of using prodrugs or prohormones to achieve supplementation of androgenic steroid concentrations in vivo also has been limited, however, in that the effectiveness of such compounds has tended to be low. In some instances, their conversion into the desired steroid is limited, for example, because they are removed from the system through the first pass effect. They also can be converted into undesirable products, for example, as in the case wherein 4-androstenedione is converted into estrogen. Even where the desired bioconversion occurs, the rate of conversion can be sufficiently low that undesirably large quantities of the prodrug must be taken to achieve desired results. This itself can have undesirable side effects.

3. Objects of the Invention

Accordingly, an object of the present invention is to provide compositions and methods that can be used to increase the in vivo concentration and bioavailability of a parent androgen.

Another object of the invention is to provide compounds and methods that can be used to increase the in vivo concentration and bioavailability of a parent androgen with relative efficiency and without requiring relatively large doses.

Additional objects and advantages of the invention will be set forth in the description that follows, and in part will be apparent from the description, or may be learned by practice of the invention. The objects and advantages of the invention may be realized and obtained by means of the instrumentalities and combinations pointed out in the appended claims.

### SUMMARY OF THE INVENTION

To achieve the foregoing objects, and in accordance with the purposes of the invention as embodied and broadly described in this document, a compound is provided for supplementing the concentration of a parent androgen in a subject in vivo. The parent androgen has a skeletal structure including a 17 position. The parent androgen also has a 17β-hydroxy group comprising a 17β-hydroxy hydrogen appended to the 17 position. “Parent androgen” as the term is used herein involves its common usage in the field, and includes but is not limited to testosterone, nandrolone, and their derivatives or analogues. The term “skeletal structure” also is used herein according to its common meaning in the field, and includes the molecular composition and structure of the parent androgen. As used herein, the skeletal structure can include the oxygen of the 17β-hydroxy group, but excludes the hydrogen of that group.

In accordance with this aspect of the invention, the compound comprises a substrate and a promoiety. The substrate has the skeletal structure of the parent androgen including a 17 position corresponding to the 17 position of the parent androgen. The promoiety comprises an alkoxymethyl ether appended to the 17 position of the substrate as a substitute for the 17β-hydroxy hydrogen.

The substrate may have the same skeletal structure as any one of a number of parent androgens. The substrate, for example, may have a structure of testosterone, nandrolone, dihydrotestosterone, dihydronandrolone, etc. The promoiety is bonded to the oxygen appended to the 17 carbon at the 17β position, and according to this aspect of the invention comprises and preferably consists of an alkoxymethyl ether. The promoiety preferably has an alkyl chain length of less than 11. The alkoxy moiety of the alkoxymethyl ether preferably consists of methoxy, but may take other forms.

In accordance with another aspect of the invention, a method is provided for increasing concentration of a parent androgen in a subject in vivo. The parent androgen has a skeletal structure including a 17 position. The parent androgen also has a 17β-hydroxy group comprising a 17β-hydroxy hydrogen appended to the 17 position.

The method according to this aspect of the invention comprises administering to the subject a compound comprising a substrate and a promoiety. The substrate has the skeletal structure of the parent androgen including a 17 position corresponding to the 17 position of the parent androgen. The promoiety comprises an alkoxymethyl ether substituted at the 17 position of the substrate for the 17β-hydroxy hydrogen. The method further includes converting the compound in vivo into the parent androgen.

In accordance with the method, the compound may be administered in a number of ways, for example, by peroral administration, pernasal administration, transdermal administration, by injecting the compound into the subject, sublingually, by combinations of these, etc. The compound also may be complexed with an hydroxypropyl beta cyclodextrin or an hydroxypropyl gamma cyclodextrin. Appropriate dosages may fall within a number of ranges, as explained more fully below, and in the presently preferred versions of the method comprise administering the compound in an amount ranging from 1.0 mg to 500 mg per day, depending on various factors.

### DETAILED DESCRIPTION OF THE PREFERRED EMBODIMENTS AND METHODS

Reference will now be made in detail to the presently preferred embodiments and methods of the invention. It should be noted, however, that the invention in its broader aspects is not limited to the specific details, representative compositions and methods, and illustrative examples described in this section in connection with the preferred embodiments and methods. The invention according to its various aspects is particularly pointed out and distinctly claimed in the attached claims read in view of this specification, and appropriate equivalents.

In accordance with one aspect of the invention, a compound is provided for supplementing the concentration of a parent androgen in a subject in vivo. As noted above, the parent androgen has a skeletal structure including a 17 position. The parent androgen also has a 17β-hydroxy group comprising a 17β-hydroxy hydrogen appended to the 17 position. The compound preferably but optionally is for treatment of a human being to supplement or increase the concentration of the parent androgen in vivo. This is not necessarily limiting, however, and veterinary applications also are possible in certain instances.

In accordance with this aspect of the invention, the compound comprises a substrate and a promoiety. The substrate has the skeletal structure of the parent androgen including a 17 position corresponding to the 17 position of the parent androgen. The promoiety comprises an alkoxymethyl ether appended to the 17 position of the substrate as a substitute for the 17β-hydroxy hydrogen.

The substrate may have the same skeletal structure as any one of a number of parent androgens. The substrate, for example, may have a structure of testosterone, nandrolone, dihydrotestosterone, dihydronandrolone, etc. Examples of parent androgens for which the substrate may have the same skeletal structure may include 5α-androst-1-ene-3α,17β-diol, androst-4-ene-3α,17β-diol, 5α-androst-1-ene-3β,17β-diol, androst-4-ene-3β,17β-diol, (5α,17β)-17-hydroxyandrost-1-ene-3-one, 17β-hydroxyandrost-4-ene-3-one,17β-hydroxyandrost-1,4-diene-3-one,5α-19-nor-androst-1-ene-3α,17β-diol, 19-nor-androst-4-ene-3α,17β-diol, 5α-19-nor-androst-1-ene-3β,17β-diol, 19-nor-androst-4-ene-3β,17β-diol,(5α,17β)-17-hydroxyestr-1-ene-3-one, 17β-hydroxyestr-4-ene-3-one,17β-hydroxyestr-1,4-diene-3-one,(5α,17β) 17-hydroxyandrostan-3-one,(5α,17β)17-hydroxyestr-3-one and mixtures or combinations thereof. Other compounds may be suitable, e.g., such as androst-1,4-diene-3α,17β-diol and androst-1,4-diene-3β,17β-diol,

The promoiety according to this aspect of the invention is bonded to the oxygen appended to the 17 carbon at the 17β position. It comprises and preferably consists of an alkoxymethyl ether. The promoiety preferably has an alkyl chain length of less than 11. The alkoxy moiety of the alkoxymethyl ether preferably consists of methoxy, but also may comprise, consist essentially of or consist of ethoxy, butoxy, isopropoxy, isobutoxy, t-butoxy, valeroxy, hexanoxy, heptanoxy, octanoxy, nonanoxy, decanoxy, undecanoxy, cyclopentoxy, cyclopentylpropoxy, and/or mixtures thereof.

The compound according to this aspect of the invention in its presently preferred embodiments may comprise (5α,17β)-17-methoxymethyloxyandrost-1-ene-3-ol,17β-methoxymethyloxyandrost-4-ene-3-ol, (5α,17β)-17-methoxymethyloxyandrost-1-ene-3-one,17β-methoxymethyloxyandrost-4-ene-3-one,17β-methoxymethyloxyandrost-1,4-diene-3-one, (5α,17β)-17-methoxymethylestra-1-ene-3-one,17β-methoxymethylestr-4-ene-3-one, (5α,17β)-17β-methoxymethylestra-1-ene-3-ol,17β-methoxymethylestr-4-ene-3-ol, (5α,17β) 17-methoxymethylandrostan-3-one, (5α,17β)17-methoxymethylestran-3-one, and mixtures or combinations thereof. In each instance where the compound includes an alcohol at the 3 position, the bond at the 3 carbon may be 3α or 3β and preferably comprises a mixture thereof.

The compound may be contained or encapsulated by an enteric coating. The compound also may be administered with a carrier, which may comprise a solid carrier, a semi-solid carrier, or a liquid carrier. A preferred liquid carrier is an aqueous emulsion including fatty acid ethyl esters, polysorbate 60, lecithin, and cholesterol or an oil.

Compounds according to this aspect of the invention may be made using known synthesis techniques. Methoxymethyl ethers of these types may be synthesized, for example, by reaction with iodomethyl methyl ether, or chloromethyl methyl ether, in a Williamson ether synthesis. The compounds may be formed by reaction of the sodium or potassium salt of the parent androgen with chloromethyl methyl ether, bromomethyl methyl ether, or iodomethyl methyl ether in dichloromethane or other suitable solvent. A 17β-methoxymethylandrost-4-ene-3-one compound, for example, synthesized in such manner typically shows under NMR spectroscopy in CDCl3 a characteristic singlet at about 5.74 parts per million (“ppm”), a triplet at 4.50 ppm, and a quadruplet at 4.17 ppm, with a melting point of about 133° C. to 135° C.

In accordance with another aspect of the invention, a method is provided for increasing concentration of a parent androgen in a subject in vivo. The parent androgen has a skeletal structure including a 17 position. The parent androgen also has a 17β-hydroxy group comprising a 17β-hydroxy hydrogen appended at the 17 position.

The method according to this aspect of the invention comprises administering to the subject a compound comprising a substrate and a promoiety. The substrate has the skeletal structure of the parent androgen including a 17 position corresponding to the 17 position of the parent androgen. The promoiety comprises an alkoxymethyl ether substituted at the 17 position of the substrate for the 17β-hydroxy hydrogen. The method further includes converting the compound in vivo into the parent androgen.

In accordance with the method, the subject may be and preferably is a human being, wherein the in vivo conversion comprises converting the compound into the parent androgen in vivo within the human being. The method may, however, be applied with respect to certain animal species.

According to preferred versions of the method, the compound administration may comprise administering the compound so that the substrate has the skeletal structure of testosterone, nandrolone, dihydrotestosterone, dihydronandrolone, and the like. This aspect of the method also may comprise administering the compound so that the substrate has the skeletal structure of the parent androgen wherein the parent androgen is selected from the group consisting of 5α-androst-1-ene-3α,17β-diol, androst-4-ene-3α,17β-diol, 5α-androst-1-ene-3β,17β-diol, androst-4-ene-3β,17β-diol,(5α,17β)-17-hydroxyandrost-1-ene-3-one, 17β-hydroxyandrost-4-ene-3-one,17β-hydroxyandrost-1,4-diene-3-one, 5α-19-nor-androst-1-ene-3α,17β-diol, 19-nor-androst-4-ene-3α,17β-diol,5α-19-nor-androst-1-ene-3β,17β-diol,19-nor-androst-4-ene-3β,17β-diol,(5α,17β)-17-hydroxyestr-1-ene-3-one, 17β-hydroxyestr-4-ene-3-one,17β-hydroxyestr-1,4-diene-3-one,(5α,17β) 17-hydroxyandrostan-3-one, (5α,17β)17-hydroxyestr-3-one and mixtures or combinations thereof. Other compounds may be suitable, e.g., such as androst-1,4-diene-3α,17β-diol and androst-1,4-diene-3β,17β-diol,

Also in accordance with this aspect of the invention, the alkoxymethyl ether as used in the method preferably has an alkyl chain length of less than 11. The alkoxy moiety of the alkoxymethyl ether preferably comprises, consists essentially or consists of methoxy. The alkoxy moiety may be selected from the group consisting of ethoxy, butoxy, isopropoxy, isobutoxy, t-butoxy, valeroxy, hexanoxy, heptanoxy, octanoxy, nonanoxy, decanoxy, undecanoxy, cyclopentoxy, cyclopentylpropoxy, and mixtures thereof. In the presently preferred version of this method, the alkoxymethyl ether comprises and more preferably consists of methoxymethyl ether.

The compound according to presently preferred version of the method may comprise (5α,17β)-17-methoxymethyloxyandrost-1-ene-3-ol,17β-methoxymethyloxyandrost-4-ene-3-ol, (5α,17β)-17-methoxymethyloxyandrost-1-ene-3-one,17β-methoxymethyloxyandrost-4-ene-3-one,17β-methoxymethyloxyandrost-1,4-diene-3-one,(5α,17β)-17-methoxymethylestra-1-ene-3-one,17β-methoxymethylestr-4-ene-3-one,(5α,17β)-17β-methoxymethylestra-1-ene-3-ol,17β-methoxymethylestr-4-ene-3-ol,(5α,17β)17-methoxymethylandrostan-3-one, (5α,17β)17-methoxymethylestran-3-one. and mixtures or combinations thereof. In each instance where the compound includes an alcohol at the 3 position, the bond at the 3 carbon may be 3α or 3β and preferably comprises a mixture thereof.

In accordance with the method, the compound may be administered by peroral administration, pernasal administration, transdermal administration, by injecting the compound into the subject, sublingually, by combinations of these, etc. If administered sublingually, it may be desirable to administer the compound after complexing it with an hydroxypropyl beta cyclodextrin and/or an hydroxypropyl gamma cyclodextrins.

The compound preferably would be administered in amounts effective to supplement or increase the concentration of the parent androgen in vivo. The appropriate dosage therefore may take into account natural or otherwise expected variations in steroid concentration, such as the normal daily variations in natural steroid production and consumption, and such as normal variations in in vivo steroid concentrations over days or weeks.

According to a related aspect of the method, the compound may be administered using a dosage given periodically for a maximum of two weeks, followed by a period, for example, of at least two weeks, of non-administration to permit recovery of natural parent androgen production in the subject, for example, to the same baseline level possessed by the subject prior to use of the compound. This can permit the compound to supplement or increase the concentration of the parent androgen in vivo for an effective period, and then terminate further dosages of the compound as its effectiveness attenuates. To provide an illustrative example, for about the first two weeks of administering compound for supplement natural testosterone such as those specifically identified above, the pituitary gland generally remains sensitized to luteinizing hormone releasing factor (“LHRH”), which is the signal from the hypothalamus for the pituitary to produce luteinizing hormone (“LH”). This in turn signals the testes to produce more testosterone. After the two-week period, for many males, the sensitivity of the pituitary to LHRH decreases, and thus continued administration of the compound would likely cause slower recovery of natural testosterone production after cessation of use. By waiting a sufficient period of time, e.g., at least about two weeks, sensitivity can be restored.

The administering step according to versions of the method also may comprise administering the compound only in daytime, i.e., during about the first hour after waking and up to about 3 hours before bed, and preferably about 6 hours before retiring. This permits the body to experience a peak blood concentration of androgen followed by a relaxation or diminution of androgen blood concentration, which has a reduced inhibitory effect on the natural production of androgen due to the cycling of blood concentration levels. This method of daytime dosing can be performed at any time during the day because it best coincides with the natural rhythm of changing androgen levels.

According to another aspect, the compound may be administered only in the morning-time, for example, within about two to three hours of waking.

The preferred dosage of the compound will depend upon the specific compound, the subject or class of subject to which it is to be administered, and other factors commonly affecting dosage determinations for this type of composition. In general, the dosage should be such that a sufficient amount of the compound enters the system of the subject and supplements or increases the natural in vivo concentration of the parent androgen. In accordance with presently preferred versions of the inventive method, the compound administration, particularly when applied to humans, comprises administering the compound in an amount ranging from 1.0 mg to 500 mg per day, more preferably in an amount ranging from 50 mg to 300 mg per day, and even more preferably in an amount ranging from 50 mg to 100 mg per day.

Additional advantages and modifications will readily occur to those skilled in the art. Therefore, the invention in its broader aspects is not limited to the specific details, representative devices and methods, and illustrative examples shown and described. Accordingly, departures may be made from such details without departing from the spirit or scope of the general inventive concept as defined by the appended claims and their equivalents.

What is claimed is:

1. A compound for supplementing the concentration of a parent androgen in a subject in vivo, the parent androgen having a skeletal structure including a 17 position and the parent androgen further having a 17β-hydroxy group comprising a 17β-hydroxy hydrogen appended to the 17 position, the compound comprising:
a substrate having the skeletal structure of the parent androgen including a 17 position corresponding to the 17 position of the parent androgen; and
a promoiety comprising an alkoxymethyl ether appended to the 17 position of the substrate as a substitute for the 17β-hydroxy hydrogen.

2. A compound as set forth in claim 1, wherein the substrate has the skeletal structure of testosterone.

3. A compound as set forth in claim 1, wherein the substrate has the skeletal structure of nandrolone.

4. A compound as set forth in claim 1, wherein the substrate has the skeletal structure of dihydrotestosterone.

5. A compound as set forth in claim 1, wherein the substrate has the skeletal structure of dihydronandrolone.

6. A compound as set forth in claim 1, wherein the substrate has the skeletal structure of the parent androgen and wherein the parent androgen is selected from the group consisting of (5α,17β)-17-hydroxy-androst-1-ene-3-one, 5α-androst-1-ene-3α,17β-diol,androst-4-ene-3α,17β-diol,5α-androst-1-ene-3β,17β-diol, androst-4-ene-3β,17β-diol, and mixtures thereof.

7. A compound as set forth in claim 1, wherein the substrate has the skeletal structure of the parent androgen and wherein the parent androgen is selected from the group consisting of 5α-19-nor-androst-1-ene-3α,17β-diol,19-nor-androst-4-ene-3α,17β-diol,5α-19-nor-androst-1-ene-3β,17β-diol, 19-nor-androst-4-ene-3β,17β-diol and mixtures thereof.

8. A compound as set forth in claim 1, wherein the alkoxymethyl ether has an alkyl chain length of less than 11.

9. A compound as set forth in claim 1, wherein the alkoxymethyl ether has an alkoxy moiety consisting of methoxy.

10. A compound as set forth in claim 1, wherein the alkoxymethyl ether has an alkoxy moiety selected from the group consisting of ethoxy, butoxy, isopropoxy, isobutoxy t-butoxy, valeroxy, hexanoxy, heptanoxy, octanoxy, nonanoxy, decanoxy, undecanoxy, cyclopentoxy, cyclopentylpropoxy, and mixtures thereof.

11. A compound as set forth in claim 1, wherein the compound comprises (5α,17β)-17-methoxymethyloxyandrost-1-ene-3-ol.

12. A compound as set forth in claim 1, wherein the compound comprises 17β-methoxymethyloxyandrost-4-ene-3-ol.

13. A compound as set forth in claim 1, wherein the compound comprises (5α,17β)-17-methoxymethyloxyandrost-1-ene-3-one.

14. A compound as set forth in claim 1, wherein the compound comprises 17β-methoxymethyloxyandrost-4-ene-3-one.

15. A compound as set forth in claim 1, wherein the compound comprises 17β-methoxymethyloxyandrost-1,4-diene-3-one.

16. A compound as set forth in claim 1, wherein the compound comprises (5α,17β)-17-methoxymethylestra-1-ene-3-one.

17. A compound as set forth in claim 1, wherein the compound comprises 17β-methoxymethylestr-4-ene-3-one.

18. A compound as set forth in claim 1, wherein the compound comprises 17β-methoxymethylestr-1-ene-3-ol.

19. A compound as set forth in claim 1, wherein the compound comprises 17β-methoxymethylestr-4-ene-3-ol.

20. A compound as set forth in claim 1, wherein the compound comprises (5α-17β)-17-methoxymethylandrostan-3-one.

21. A compound as set forth in claim 1, wherein the compound comprises (5α-17β)-17-methoxymethylestran-3-one.

22. A compound as set forth in claim 1, further including a carrier.

23. A compound as set forth in claim 1, wherein the carrier comprises a solid carrier.

24. A compound as set forth in claim 1, wherein the carrier comprises a liquid carrier.

25. A compound as set forth in claim 1, wherein the carrier comprises a semi-solid carrier.

26. A method for increasing concentration of a parent androgen in a subject in vivo, the parent androgen having a skeletal structure including a 17 position and the parent androgen further having a 17β-hydroxy group comprising a 17β-hydroxy hydrogen appended at the 17 position, the method comprising:
administering to the subject a compound comprising a substrate and a promoiety, the substrate having the skeletal structure of the parent androgen including a 17 position corresponding to the 17 position of the parent androgen, and the promoiety comprising an alkoxymethyl ether substituted at the 17 position of the substrate for the 17β-hydroxy hydrogen; and
converting the compound in vivo into the parent androgen.

27. A method as set forth in claim 26, wherein the subject is a human being and the in vivo conversion comprises converting the compound into the parent androgen in vivo within the human being.

28. A method as set forth in claim 26, wherein the compound administration comprises administering the compound so that the substrate has the skeletal structure of testosterone.

29. A method as set forth in claim 26, wherein the compound administration comprises administering the compound so that the substrate has the skeletal structure of nandrolone.

30. A method as set forth in claim 26, wherein the compound administration comprises administering the compound so that the substrate has the skeletal structure of dihydrotestosterone.

31. A method as set forth in claim 26, wherein the compound administration comprises administering the compound so that the substrate has the skeletal structure of dihydronandrolone.

32. The method as set forth in claim 26, wherein the compound administration comprises administering the compound so that the substrate has the skeletal structure of the parent androgen wherein the parent androgen is selected from the group consisting of (5α,17β)-17-hydroxy-androst-1-ene-3-one,
5α-androst-1-ene-3α,17β-diol, androst-4-ene-3α,17β-diol, 5α-androst-1-ene-3β,17β-diol, androst-4-ene-3β,17β-diol, and mixtures thereof.

33. A method as set forth in claim 26, wherein the compound administration comprises administering the compound so that the substrate has the skeletal structure of at least one of 5α-19-nor-androst-1-ene-3β,17β-diol, 19-norandrost-4-ene-3α-17β-diol,5α-19-nor-androst-1-ene-3β,17β-diol,19-nor-androst-4-ene-3β,17β-diol.

34. A method as set forth in claim 26, wherein the alkoxymethyl ether has an alkyl chain length of less than 11.

35. A method as set forth in claim 26, wherein the alkoxymethyl ether has an alkoxy moiety consisting of methoxy.

36. A method as set forth in claim 26, wherein the alkoxymethyl ether has an alkoxy moiety selected from the group consisting of ethoxy, butoxy, isopropoxy, and isobutoxy, t-butoxy, valeroxy, hexanoxy, heptanoxy, octanoxy, nonanoxy, decanoxy, undecanoxy, cyclopentoxy, cyclopentylpropoxy, and mixtures thereof.

37. A method as set forth in claim 26, wherein the alkoxymethyl ether comprises methoxymethyl ether.

38. A method as set forth in claim 26, wherein the compound comprises 17β-methoxymethyloxyandrost-1-ene-3-ol.

39. A method as set forth in claim 26, wherein the compound comprises (5α,17β)-17-methoxymethyloxyandrost-4-ene-3-ol.

40. A method as set forth in claim 26, wherein the compound comprises (5α,17β)-17-methoxymethyloxyandrost-1-ene-3-one.

41. A method as set forth in claim 26, wherein the compound comprises 17β-methoxymethyloxyandrost-4-ene-3-one.

42. A method as set forth in claim 26, wherein the compound comprises 17β-methoxymethyloxyandrost-1,4-diene, 3-one.

43. A method as set forth in claim 26, wherein the compound comprises (5α,17β)-17β-methoxymethylestr-1-ene-3-one.

44. A method as set forth in claim 26, wherein the compound comprises 17β-methoxymethylestr-4-ene-3-one.

45. A method as set forth in claim 26, wherein the compound comprises 17β-methoxymethylestr-1-ene-3-ol.

46. A method as set forth in claim 26, wherein the compound comprises 17β-methoxymethylestr-4-ene-3-ol.

47. A method as set forth in claim 26, wherein the compound comprises (5α-17β)-17-methoxymethylandrostan-3-one.

48. A method as set forth in claim 26, wherein the compound comprises (5α-17β)-17-methoxymethylestran-3-one.

49. A method as set forth in claim 26, wherein the compound administration comprises peroral administration.

50. A method as set forth in claim 26, wherein the compound administration comprises pernasal administration.

51. A method as set forth in claim 26, wherein the compound administration comprises transdermal administration.

52. A method as set forth in claim 26, wherein the compound administration comprises injecting the compound into the subject.

53. A method as set forth in claim 26, wherein the compound administration comprises administering the compound sublingually.

54. A method as set forth in claim 26, wherein the compound administration comprises complexing the compound with an hydroxypropyl beta cyclodextrin.

55. A method as set forth in claim 26, wherein the compound administration comprises complexing the compound with an hydroxypropyl gamma cyclodextrin.

56. A method as set forth in claim 26, wherein the compound administration comprises administering a dosage periodically for a maximum of two weeks, followed by at least two weeks of non-administration to permit recovery of natural parent androgen production in the subject.

57. A method as set forth in claim 26, wherein the compound administration comprises administering the compound only in morning-time.

58. A method as set forth in claim 26, wherein the compound administration comprises administering the compound in an amount ranging from 1.0 mg to 500 mg per day.

59. A method as set forth in claim 26, wherein the compound administration comprises administering the compound in an amount ranging from 50 mg to 300 mg per day.

60. A method as set forth in claim 26, wherein the compound administration comprises administering the compound in an amount ranging from 50 mg to 100 mg per day.

61. A method as set forth in claim 26, wherein the compound administration further includes applying an enteric coating to the compound prior to administering the compound.
